(19) United States
(12) Patent Application Publication
(10) Pub. No.: US 2026/0176668 A1
(43) Pub. Date: Jun. 25, 2026

(54) CULTURE MEDIUM FOR DETECTING MICROORGANISMS COMPRISING A MIXTURE OF AGAR AND KAPPA-CARRAGEENAN AS GELLING AGENT

(71) Applicant: bioMérieux, Marcy L'Etoile (FR)

(72) Inventors: Bosi MAO, Lyon (FR); Laurent LEBLANC, Genay (FR); Vivian SCHNEIDER, Chazay d'Azergues (FR)

(73) Assignee: bioMérieux, Mercy L'Etoile (FR)

(21) Appl. No.: 19/128,209

(22) PCT Filed: Nov. 7, 2023
(86) PCT No.: PCT/EP2023/081050

§ 371 Date: May 8, 2025

(30) Foreign Application Priority Data
Nov. 10, 2022 (EP) 22206665.6

(51) Int. Cl. C12Q 1/04

(52) U.S. Cl. CPC C12Q 1/045

(57) ABSTRACT

A culture medium relating to the field of molecular biology and more particularly that of clinical and industrial microbiology, and for detecting microorganisms, including, as a gelling agent, a mixture of agar and kappa-carrageenan.

### TECHNICAL FIELD

The present invention relates to the field of microbiology and more particularly to that of industrial and clinical microbiology. More specifically, the present invention relates to a novel culture medium for detecting microorganisms, comprising a mixture of agar and κ-carrageenan as gelling agent.

### PRIOR ART

The detection and identification of microorganisms is very important in both clinical and industrial fields. In the pharmaceutical and agrifood industries, culture media are still the benchmark for detecting and counting microorganisms. The agrifood industry uses these products for microbiological controls on products intended for human and animal consumption. In the pharmaceutical industry, media are mainly used to perform environmental controls (air, surface, operator, etc.) and to check that there is no bacterial contamination in products during manufacture. In many cases, the controls performed are also taken into consideration when releasing batches of finished or semifinished products. Many tests and procedures depend on the ability of culture media to give homogeneous, reproducible results. Media requirements may be specific to both the sample and the strains to be tested. Culture media meeting established performance criteria are therefore a prerequisite for any reliable microbiological analysis. Ready-to-use media are manufactured under standardized production conditions and solve many problems in microbiology laboratories where time, equipment or trained personnel are often lacking. Thus, ready-to-use culture media of recognized quality enable reliable microbiological analysis.

They may be liquid, solid or semi-solid media supplied in dishes, vials, tubes or other containers.

A major component of solid or semi-solid culture media is the gelling agent, notably agar.

Agar is extracted from red algae of the Rhodophyte family. Agar has the drawback of being derived from a limited natural resource. Thus, Lines (Applied and Environmental Microbiology, December 1977, pages 637-639) describes the use of κ-carrageenans as a substitute for agar in a culture medium. The drawback of a culture medium in which the agar has been substituted with κ-carrageenan is that it begins to solidify at high temperatures. Whereas agar is dispensed at a temperature of approximately 45-55° C., a κ-carrageenan-based culture medium requires a temperature above 60° C. to be dispensed into dishes. This leads to problems of industrialization of agar-based culture media, with the need to maintain a high temperature throughout the manufacturing process, or the risk of burns to technicians if the Petri dishes are poured at the place of use. Furthermore, a high gelling temperature is not compatible with the production of culture media containing heat-sensitive compounds such as antibiotics or blood.

On the other hand, the gelling agent is also the key compound for improving the shelf life of culture media. Agar-based culture media, although widely used, have drawbacks, for instance syneresis (release of water from the gel network) and shrinkage, which should be avoided. Thus, WO 2004/050675 describes the use of a mixture of agar and iota-carrageenan in an agar-based culture medium. This mixture limits gel syneresis, thus improving its stability and shelf life.

Nevertheless, there is still a need to develop an agar-based culture medium offering physico-chemical parameters and resistance to shrinkage that will improve its shelf life while at the same time maintaining optimum quality and remaining compatible with industrialization.

### SUMMARY OF THE INVENTION

One aim of the present invention is to provide a culture medium that is dehydration-resistant due to the fact that it displays little or no shrinkage.

Another aim of the invention is to provide a culture medium with a gelling temperature that is compatible with the industrialization or use of heat-sensitive compounds.

These and other objectives are achieved by the present invention, which relates to a culture medium for detecting microorganisms, comprising
agar or agarose,
κ-carrageenan,
   the weight ratio [agar, κ-carrageenan]:[other gelling agent] being greater than 70:30.

Entirely advantageously, the weight ratio of agar:κ-carrageenan in the culture medium is between 80:20 and 30:70.

In one particular embodiment of the invention, when the concentration of salts present in the medium is less than 5 g/l, the weight ratio of agar:κ-carrageenan is between 80:20 and 60:40.

In another particular embodiment of the invention, when the concentration of salts present in the medium is between 5 g/l and 20 g/l, the weight ratio of agar:κ-carrageenan is between 80:20 and 30:70.

In another particular embodiment of the invention, when the concentration of salt present in the medium is greater than 20 g/l, the weight ratio of agar:κ-carrageenan is between 80:20 and 60:40.

Advantageously, the culture medium also includes a heat-sensitive compound, such as an antibiotic and/or blood.

Advantageously, the culture medium is gelled in a Petri dish.

Another subject of the invention relates to a process for obtaining a medium according to the invention, characterized in that it consists essentially in:
supercooling the compounds in the culture medium to mix them
cooling to a first temperature above the gelling temperature of the culture medium
filling Petri dishes with this culture medium in solution
allowing to cool to reach the gelling temperature to form a gelled culture medium, the gelling temperature being between 30° C. and 45° C.

Another subject of the invention relates to a culture medium comprising an agar: κ-carrageenan mixture having a weight ratio of between 80:20 and 30:70 and a gelling temperature of between 30° C. and 45° C.

Another subject of the invention relates to an in vitro microbiological culture method, in which microorganisms that are liable to be present in a sample are inoculated in or on a culture medium according to the invention.

Another subject of the invention relates to a method for detecting a target microorganism in a sample liable to contain same, involving the following steps:
placing said sample in contact with a gelled culture medium according to the invention
incubating
detecting the presence of said microorganism.

### DESCRIPTION OF THE FIGURES

FIG. 1 is a diagram representing the percentage of intact (non-shrunken) culture medium after the Extreme Dehydration Test. Series “a” media comprise 100% agar (or agarose for medium 1). Series “b” media comprise a ratio of agar:κ-carrageenan of 50:50 (or a ratio of agarose:κ-carrageenan of 50:50 for medium 1).

Source of agar (or agarose for medium 1):
Medium 1a and medium 1b: Agarose from Sigma-Aldrich
Medium 2a and medium 2b: Agar from Sigma-Aldrich
Medium 3a and medium 3b: American agar from Roko
Medium 4a and medium 4b: European agar from Roko
Medium 5a and medium 5b: European agar from Setexam
Medium 6a and medium 6b: Gracilaria agar from Roko

FIG. 2 represents photos of the culture media after the Extreme Dehydration Test. Series “a” media comprise 100% agar (or agarose for medium 1). Series “b” media comprise a ratio of agar:κ-carrageenan of 50:50 (or a ratio of agarose:κ-carrageenan of 50:50 for medium 1). Source of agar (or agarose for medium 1):
Medium 1a and medium 1b: Agarose from Sigma-Aldrich
Medium 2a and medium 2b: Agar from Sigma-Aldrich
Medium 3a and medium 3b: American agar from Roko
Medium 4a and medium 4b: European agar from Roko
Medium 5a and medium 5b: European agar from Setexam
Medium 6a and medium 6b: Gracilaria agar from Roko

FIG. 3 represents photos of mixed agar culture media with different carrageenans after the Extreme Dehydration Test.
Medium 1 comprises 100% European agar (Setexam)
Medium 2: comprises a ratio of agar:κ-carrageenan (Sigma-Aldrich) of 50:50
Medium 3 comprises a ratio of agar:κ-carrageenan (Setexam) of 50:50
Medium 4 comprises a ratio of agar:κ-carrageenan (Roko) of 50:50
Medium 5 comprises a ratio of agar:λ-carrageenan (Sigma-Aldrich) of 50:50
Medium 6 comprises a ratio of agar:ι-carrageenan (Sigma-Aldrich) of 50:50.

FIG. 4 represents the percentage of non-shrunken culture media for different ratios of agar:κ-carrageenan after the Extreme Dehydration Test.
Medium 1 comprises 100% European agar
Medium 2 comprises a ratio of agar:κ-carrageenan of 70:30
Medium 3 comprises a ratio of agar:κ-carrageenan of 60:40
Medium 4 comprises a ratio of agar:κ-carrageenan of 50:50
Medium 5 comprises a ratio of agar:κ-carrageenan of 30:70
Medium 6 comprises a ratio of agar:κ-carrageenan of 0:100.

FIG. 5 (a) represents the gelling temperature of TSA media modified with different ratios of agar: κ-carrageenan.
Medium 1 comprises 100% agar
Medium 2 comprises a ratio of agar:κ-carrageenan of 70:30
Medium 3 comprises a ratio of agar:κ-carrageenan of 50:50
Medium 4 comprises a ratio of agar:κ-carrageenan of 30:70
Medium 5 comprises a ratio of agar:κ-carrageenan of 0:100.

FIG. 5 (b) represents the viscosity of TSA media modified with different ratios of agar: κ-carrageenan.
Medium 1 comprises 100% agar
Medium 2 comprises a ratio of agar:κ-carrageenan of 70:30
Medium 3 comprises a ratio of agar:κ-carrageenan of 50:50
Medium 4 comprises a ratio of agar:κ-carrageenan of 30:70
Medium 5 comprises a ratio of agar:κ-carrageenan of 0:100.

FIG. 6 represents the gelling temperature of Chapman media modified with different ratios of agar: κ-carrageenan.
Medium 1 comprises 100% agar
Medium 2 comprises a ratio of agar:κ-carrageenan of 80:20
Medium 3 comprises a ratio of agar:κ-carrageenan of 70:30
Medium 4 comprises a ratio of agar:κ-carrageenan of 60:40
Medium 5 comprises a ratio of agar:κ-carrageenan of 50:50

Medium 6 comprises a ratio of agar:κ-carrageenan of 0:100.

FIG. 7(a) represents the gelling temperature of SDA media modified with different ratios of agar: κ-carrageenan.
Medium 1 comprises 100% agar
Medium 2 comprises a ratio of agar:κ-carrageenan of 80:20
Medium 3 comprises a ratio of agar:κ-carrageenan of 70:30
Medium 4 comprises a ratio of agar:κ-carrageenan of 60:40
Medium 5 comprises a ratio of agar:κ-carrageenan of 50:50
Medium 6 comprises a ratio of agar:κ-carrageenan of 30:70
Medium 7 comprises a ratio of agar:κ-carrageenan of 0:100.

FIG. 7 (b) represents the elastic modulus of SDA media modified with different ratios of agar: κ-carrageenan.
Medium 1 comprises 100% agar
Medium 2 comprises a ratio of agar:κ-carrageenan of 80:20
Medium 3 comprises a ratio of agar:κ-carrageenan of 70:30
Medium 4 comprises a ratio of agar:κ-carrageenan of 60:40
Medium 5 comprises a ratio of agar:κ-carrageenan of 50:50
Medium 6 comprises a ratio of agar:κ-carrageenan of 30:70
Medium 7 comprises a ratio of agar:κ-carrageenan of 0:100.

FIG. 8 shows photos after incubation of culture media inoculated with various microorganisms.

Line (a) corresponds to a TSA medium comprising 100% agar and line (b) corresponds to a modified TSA medium comprising a ratio of agar:κ-carrageenan of 70:30.
Column 1 corresponds to inoculation with S. aureus
Column 2 corresponds to inoculation with B. subtilis
Column 3 corresponds to inoculation with P. aeruginosa
Column 4 corresponds to inoculation with C. albicans
Column 5 corresponds to inoculation with A. brasiliensis
Column 6 corresponds to inoculation with E. coli

FIG. 9 shows photos of Chapman culture media modified after incubation and inoculated with Staphylococcus aureus.

Medium 1 corresponds to a medium with 100% agar.
Medium 2 comprises a ratio of agar:κ-carrageenan of 80:20.
Medium 3 comprises a ratio of agar:κ-carrageenan of 50:50.

### DETAILED DESCRIPTION OF THE INVENTION

Certain terms and expressions used in the context of the invention are detailed hereinbelow.

A first subject of the invention relates to a culture medium for detecting microorganisms, comprising
agar or agarose
κ-carrageenan,
   the weight ratio [agar, κ-carrageenan]:[other gelling agent] being greater than 70:30.

The culture medium according to the invention also comprises a peptone as a source of amino acids and nitrogen.

The term “culture medium” means a medium comprising all the elements required for the growth of microorganisms. In general, the agar-based culture medium comprises:
a source of carbon, generally provided by sugars
a source of amino acids and nitrogen provided by peptones
various salts and buffers
demineralized water
a gelling agent

Other ingredients may be added, for example selective agents. The medium may also comprise a colorant.

According to the present invention, the culture medium may be in the form of a ready-to-use gel, i.e. ready for inoculation in a Petri dish. This is also referred to as a gelled culture medium, or a medium in solid form. The culture medium may also be in powder form. In this case, it is then rehydrated, heated/sterilized and then poured into a Petri dish to form a ready-to-use gelled medium. Agar is the traditional gelling agent used in microbiology for culturing microorganisms, but other gelling agents can also be used, for example gellan gum, pectin, guar gum, gelatin, and also other natural or artificial gelling agents.

Thus, according to the present invention, the weight ratio [agar, κ-carrageenan]:[other gelling agent] is greater than 70:30. Preferentially, the weight ratio [agar, κ-carrageenan]:[other gelling agent] is greater than 80:20, even more preferentially greater than 90:10, even more preferentially greater than 95:5.

The ratio [agar, κ-carrageenan]:[other gelling agent] may be 100:0.

Agar is an unbranched polysaccharide obtained from the cell walls of certain species of red algae, mainly tengusa (Gelidiaceae) and ogonori (Gracilaria). Agar provides a sufficiently stable network suitable for bacterial proliferation without serving as food for them, preserving the matrix structure of the gelling agent. A number of preparations are commercially available, for example Columbia agar, Trypcase soy agar, MacConkey agar, Mueller Hinton agar or, more generally, those described in the Handbook of Microbiological Media.

According to the present invention, the culture medium may comprise agarose, which is a polysaccharide extracted from agar, composed of the repetition of a diholoside. Agarose is the gelling part of agar.

According to the present invention, the culture medium comprises κ-carrageenan. Carrageenans are anionic polysaccharides extracted from red algae, mainly Gigarina, Chondrus and Eucheuma. They exist in several forms, and the main commercial classes are:
The κ-carrageenan form, a rigid gel in the presence of monovalent ions such as potassium, sodium, rubidium, lithium, etc, or divalent ions such as calcium or magnesium. It is mainly derived from the alga Kappaphycus alvarezii.
The ι-carrageenan form, a soft gel in the presence of calcium ions. It is mainly derived from the alga Eucheuma denticulatum.
The λ-carrageenan form, which does not form a gel.

Surprisingly, a culture medium comprising a mixture of agar and κ-carrageenan was found to improve the medium's resistance to shrinkage. Specifically, culture media, in their routine use, will be incubated at temperatures of between 25 and 45° C. for periods that may in some cases be as long as 28 days. For pharmaceutical environmental control applications, agar media are exposed for several hours to dehydrating laminar air flows. In all these cases of use, culture media undergo severe dehydration, leading to destabilization of the agar network and the appearance of tensions within the gel. For culture media containing agar, this phenomenon may lead to shrinkage, cracking and/or detachment. These defects have a direct impact on the microbiological performance of the media. Technical solutions to limit their appearance despite dehydration represent a major advantage in the quality of the results delivered by these products. Furthermore, greater resistance to agar shrinkage means longer shelf-lives.

Preferentially, the concentration of the agar and κ-carrageenan mixture is between 10 g/l and 30 g/l, preferentially between 10 g/l and 20 g/l. Even more preferentially between 13 g/l and 17 g/l. The agar and κ-carrageenan mixture is the predominant gelling agent in the culture medium according to the invention. It is the main gelling agent. Thus, the medium according to the invention comprises a mixture of agar and κ-carrageenans in a weight ratio [agar, κ-carrageenan]:[other gelling agent] of greater than 70:30, even more preferentially greater than 80:20, even more preferentially greater than 90:10, even more preferentially greater than 95:5. The weight ratio [agar, κ-carrageenan]:[other gelling agent] may be 100:0.

Another gelling agent may be added in a minor proportion to the medium according to the invention if the shrinkage resistance of said medium remains greater than that of a medium comprising no gelling agent other than agar at the same concentration of gelling agents. For example, mention may be made of gelatin, pectin and guar gum. Thus, a medium according to the invention may include 15 g/l of a mixture of agar and κ-carrageenans supplemented with 2 g/l of guar gum, with improved shrinkage behavior relative to a medium containing 17 g/l of agar alone.

Advantageously, the weight ratio of agar:κ-carrageenan is between 80:20 and 30:70.

The term “shrinkage” refers to the partial or total detachment of the culture medium from the side wall of the Petri dish. This then leads to a reduction in surface area. The shrinkage resistance may be measured by the “Extreme Dehydration Test”, which involves exposing the dishes to a vertical laminar airflow hood for 60 hours without a lid. The culture media will dehydrate during exposure. The surface area of each dehydrated medium is then measured using a dish-reading instrument such as Scan® 4000. A culture medium with low shrinkage resistance will rapidly peel away from the side wall of the dish during the test, and will continue to shrink until a completely dry fraction of the initial surface is obtained. On the other hand, a culture medium with good shrinkage resistance will not peel away from the side wall of the dish until later, if at all, during the test, and the surface area of the dried medium will decrease only slightly, if at all.

The agar:κ-carrageenan ratio also has an effect on agar gelation. These properties are measured by agar's gelling temperature, viscosity and elastic modulus.

The gelling temperature is measured using a rheometer. Samples of liquid culture media are placed on the rheometer's Peltier plate, preheated to a temperature higher than the sample's gelling temperature. The samples are then cooled to 20° C., with a cooling rate of 3° C./min, similar to that used in production. During cooling, an oscillatory strain is imposed on the sample, enabling the study of the viscoelastic properties and changes from “liquid” to “solid” phases of the sample. The gelling temperature is defined by a rapid increase in elastic modulus or viscosity, representing the start of gelling agent networks and solidification of the medium.

The gelling temperature may also depend on the physicochemical conditions of the medium, such as ionic charge or pH. Some media contain high concentrations of salts (potassium, sodium, calcium), as do some selective media. However, the presence of ions increases the gelling temperature. A gelling temperature of between 3° and 45° C. is particularly advantageous, as it enables the medium to be produced in a readily industrializable manner. Specifically, it will not be necessary to maintain the medium at a high temperature to prevent it from gelling before being poured into dishes. Furthermore, if the medium is prepared in the laboratory, a temperature of less than 60° C. will prevent burns. Moreover, such a medium also allows the addition of heat-sensitive compounds such as blood or antibiotics, which are then not deteriorated by heat.

The viscosity is measured with a rheometer. Viscosity is a fluid's resistance to change of shape. It determines the fluid's speed of movement. The more viscous the fluid, the slower the movement. For culture media with an agar:κ-carrageenan mix, a viscosity similar to that of a medium with 100% agar is particularly advantageous, as it enables production of the medium without the need to modify the parameters of the manufacturing process, i.e. stirring speed or pump pressure for distribution.

The elastic modulus is an intrinsic quantity of a material, defined by the ratio of a stress to the elastic strain caused by this stress. For culture media, it represents the hardness of the medium. The softer the medium, the lower the elastic modulus. A culture medium with an elastic modulus greater than 5 kPa is required for correct manual or automatic inoculation. It is measured using a rheometer. Samples of liquid culture media are placed on the rheometer's Peltier plate, preheated to a temperature higher than the sample's gelling temperature. The samples are then cooled to 20° C., at a cooling rate of 3° C./min, similar to that used in production. During cooling, an oscillatory strain is imposed on the sample, enabling the study of the viscoelastic properties and changes from “liquid” to “solid” phases of the sample. During cooling, the elastic modulus increases as the medium gels. It then reaches a stable value, indicating the completion of gelation. This final value corresponds to the elastic modulus of the medium.

Thus, in one particular embodiment of the invention, the medium has a salt concentration of less than 5 g/l and a weight ratio of agar:κ-carrageenan of between 80:20 and 60:40. Media with a salt concentration of less than 5 g/l are well known to those skilled in the art. For example, Sabouraud Dextrose agar medium may be mentioned. Substituting the gelling agent of a medium with a low ionic load with a mixture of agar:κ-carrageenan having a ratio of between 80:20 and 60:40, makes it possible to obtain a medium with an appropriate elastic modulus, i.e. greater than 5 kPa, making it easier to inoculate. It also affords a gelling temperature of between 30° C. and 45° C., and a viscosity similar to that of an unmodified medium with 100% agar, making it easier to industrialize.

This ratio of between 80:20 and 60:40 is also particularly suitable for a medium with a high salt concentration in excess of 20 g/l. Thus, in one particular embodiment of the invention, the medium has a salt concentration of greater than 20 g/l and the weight ratio of agar:κ-carrageenan is between 80:20 and 60:40. Media with a high salt concentration, i.e. greater than 20 g/l, are well known to those skilled in the art. For example, Chapman medium may be mentioned. Substituting the gelling agent of a medium with a high salt concentration for a mixture of agar:κ-carrageenan having a ratio of between 80:20 and 60:40 affords an appropriate elastic modulus, i.e. greater than 5 kPa, making it easier to inoculate. It also has a gelling temperature of between 30° C. and 45° C., and a viscosity similar to that of an unmodified medium with 100% agar, making it easy to industrialize.

In another particular embodiment of the invention, the medium according to the invention has a salt concentration of between 5 g/l and 20 g/l, and a weight ratio of agar:κ-carrageenan of between 80:20 and 30:70. Media with a conventional salt concentration, i.e. between 5 and 20 g/l, are well known to those skilled in the art. For example, mention may be made of the TSA medium. Substituting the gelling agent in a medium with a salt concentration of between 5-20 g/l for a mixture of agar:κ-carrageenan with a ratio of 80:20-30:70, produces a medium with an elastic modulus that is compatible with inoculation, i.e. greater than 5 kPa. This ratio also makes it possible to obtain a gelling temperature of between 30° C. and 45° C., and a viscosity similar to that of an unmodified medium with 100% agar, making it easier to industrialize.

In a preferred embodiment, the culture medium also comprises a heat-sensitive compound. The heat-sensitive compound may be an antibiotic and/or blood. Thus, a culture medium according to the invention with a gelling temperature of between 30° C. and 45° C. enables the preservation of heat-sensitive compounds.

Another subject of the invention relates to a process for obtaining a medium, characterized in that it consists essentially in:
supercooling the compounds in the culture medium to mix them
cooling to a first temperature above the gelling temperature of the culture medium
filling Petri dishes with this culture medium in solution
allowing to cool to reach the gelling temperature to form a gelled culture medium, the gelling temperature being between 30° C. and 45° C.

In general, the compounds in the medium are supercooled at a temperature above 80° C., or even above 100° C., to enable their sterilization.

The compounds are then mixed. The supercooled culture medium is then cooled to a temperature that remains above the gelling temperature of the culture medium. In general, this temperature is between 5° and 60° C.

If heat-sensitive compounds are required, they are then added.

The culture medium is then poured into the Petri dishes. It then reaches its gelling temperature, which is the temperature characterizing the phase transition from “liquid” to “solid”.

Another subject of the invention relates to a culture medium comprising an agar: κ-carrageenan mixture having a weight ratio of between 80:20 and 30:70 and a gelling temperature of between 30° C. and 45° C., the weight ratio [agar, κ-carrageenan]:[other gelling agent] being greater than 70:30.

This medium then constitutes an intermediate product in the process for obtaining the medium when its temperature reaches the temperature for phase transition from “liquid” to “solid”.

Another subject of the invention relates to a medium according to the invention gelled in a Petri dish.

Another subject of the invention relates to an in vitro microbiological culture method, in which microorganisms that are liable to be present in a sample are inoculated in or on a culture medium according to the invention.

Another subject of the invention relates to a method for detecting a target microorganism in a sample liable to contain same, involving the following steps:
placing said sample in contact with a gelled culture medium according to the invention
incubating
detecting the presence of said microorganism.

The present invention is illustrated in a nonlimiting manner by the following examples.

### EXAMPLES

### Example 1: Preparation of Culture Media According to the Invention and Other Culture Media

Different concentrations of gelling agents (agar, carrageenan) were used: from 0 to 15 g/l. Different weight ratios of agar: carrageenan were used to make up the agar: carrageenan gelling mix:
0:100; 30:70; 40:60; 50:50; 70:30; 20:80; 100:0.

Different media, called modified media, were prepared from a base of TSA, Chapman or Sabouraud Dextrose Agar media (tables below) in which the agar was replaced with an agar:carrageenan mix, the ratios of which are given in the examples.

TABLE 1
Formulation of modified TSA medium

| | | |
|---|---|---|
| Casein peptone (bovine) | 15 | g |
| Soybean peptone | 5 | g |
| Yeast extract | 6 | g |
| Sodium chloride | 5 | g |
| Sodium pyruvate | 2 | g |
| Soya lecithin | 0.7 | g |
| Polysorbate 80 | 5 | g |
| Sodium thiosulfate 5H2O | 0.05 | g |
| L-Histidine | 1 | g |
| Gelling agent | 15 | g |
| Purified water | 1 | L |

pH 7.3

TABLE 2
Formulation of modified Chapman medium

| | | |
|---|---|---|
| Meat extract (bovine or porcine) | 1 | g |
| Casein peptone (bovine or porcine) | 5 | g |
| Meat peptone (bovine or porcine) | 5 | g |
| Sodium chloride | 75 | g |
| D-Mannitol | 10 | g |
| Gelling agent | 15 | g |
| Phenol red | 25 | mg |
| Purified water | 1 | L |

pH 7.4

TABLE 3
Formulation of modified Sabouraud Dextrose Agar medium

| | | |
|---|---|---|
| Dextrose | 15 | g |
| Pancreatic digestion of casein (bovine) | 5 | g |
| Pancreatic digestion of animal tissue | 5 | g |
| (bovine or porcine) | | |
| Gelling agent | 15 | g |
| Purified water | 1 | L |

pH 5.6

The preparation involves the following steps:
1) The agars, carrageenans and other starting materials are weighed out according to the formulation for each culture medium.
2) All the starting materials are diluted with demineralized water.
3) The resulting mixture is stirred and heated to the boiling point to dissolve the starting materials completely.
4) The solution is autoclaved in a liquid cycle, with a plateau at 120° C. for 16 min. Steps 3) and 4) may also be performed using an automated culture media preparation instrument such as Masterclave®.
5) 20-30 ml of medium are poured hot, i.e. at a temperature above the gelling temperature of the medium, into 90 mm Petri dishes without lids.
6) The lids are placed on the dishes.
7) The culture media dishes are stored at 2-8° C.

### Example 2: Shrinkage-Resistance Testing of Media According to the Invention Comprising Agar from Different Suppliers

Different types of agar were tested for shrinkage resistance:
European Agar (Roko; ref 03904182, Batch 180100340)
European agar (Setexam; ref 03904185, Batch M2019)
American Agar (Roko; ref 3904170, Batch 200201654)
Commercial agar (Sigma-Aldrich, ref 05040 CAS: 9002-18-0, Batch BCCF9819)
Gracilaria Agar (Roko, ref Rokoagar RGM LAB, Batch 200201758)
Agarose (Sigma-Aldrich, ref A4718 CAS: 9012-36-6, Batch SLCK4183)

The media tested included either agar alone or as a mixture with κ-carrageenan from the supplier Setexam (Setexam, Reference Danish agar: 03904187, Batch C19152).

For the media with agar alone, the agar concentration is 15 g/1.

For the media with an agar:κ-carrageenan mix, a 50:50 ratio was used. The total gelling agent concentration was 15 g/l.

The TSA formulation was used for this test. The culture media were prepared using a Masterclave®. 30 ml of medium were added to each 90 mm Petri dish.

The Extreme Dehydration Test method was used to assess the shrinkage resistance of each medium in Petri dishes:
Thirty dishes of each formulation were exposed under a vertical laminar airflow hood (V=0.45 m/s) for 60 hours without a cover.
The culture medium was completely dehydrated after exposure, with total water loss.
The appearance of each culture medium was observed post-exposure
The surface area of each dried culture medium was measured using a Scan® 4000 automatic colony counter (ref. 438000, supplier Interscience).

The results were presented as a percentage, calculated relative to the initial surface area of the culture medium.

TABLE 4
Results of the TSA Extreme Dehydration Test method with
an agar:κ-carrageenan mix comprising different agars.

| | % Surface area of dried medium/surface | |
|---|---|---|
| | area of initial medium | |
| | <a> | <b> |
| | Agar:κ-carrageenan = | Agar:κ-carrageenan = |
| | 100:0 | 50:50 |
| 1 Agarose -Sigma- | 25.9 | 82.8 |
| Aldrich | | |
| 2 Agar -Sigma- | 42.9 | 88.1 |
| Aldrich | | |
| 3 Agar | 46.1 | 87.1 |
| American -Roko | | |
| 4 European | 46.5 | 91.9 |
| Agar -Roko | | |
| 5 European | 48.9 | 89.1 |
| Agar -Setexam | | |
| 6 Gracilaria | 60.0 | 90.6 |
| Agar -Roko | | |

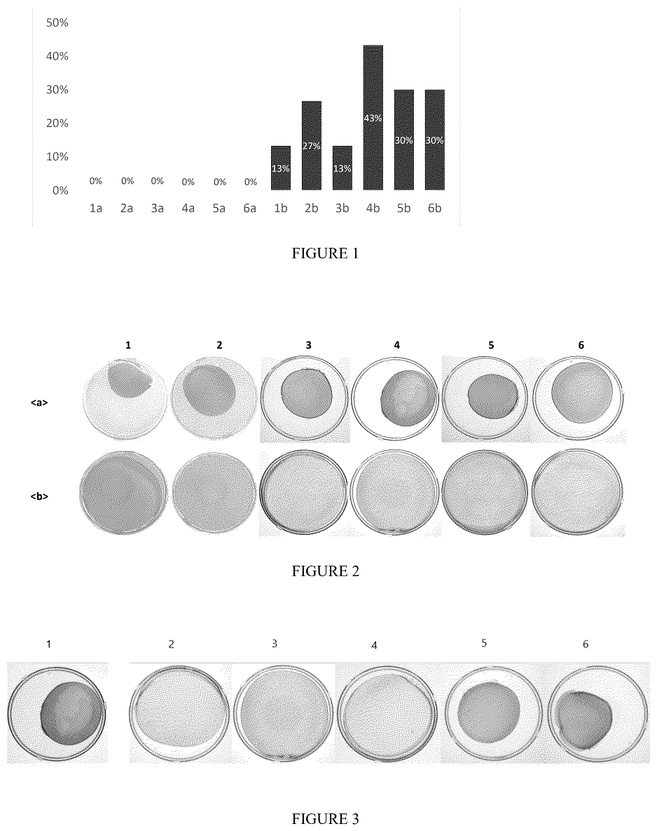

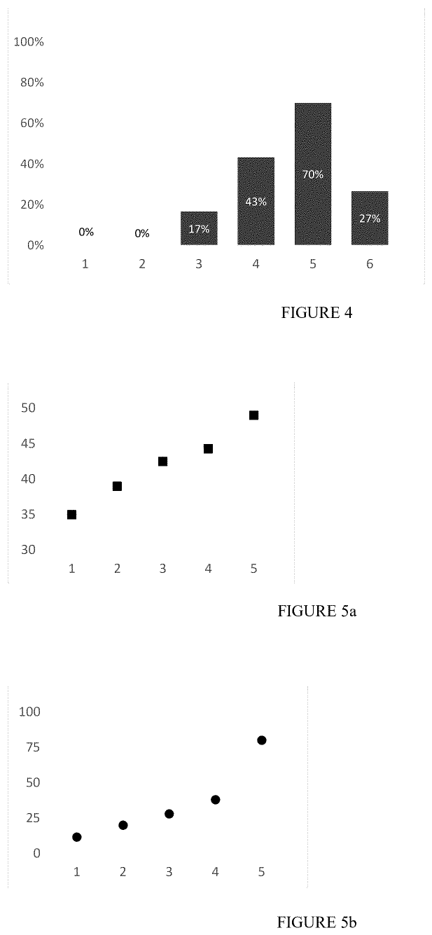

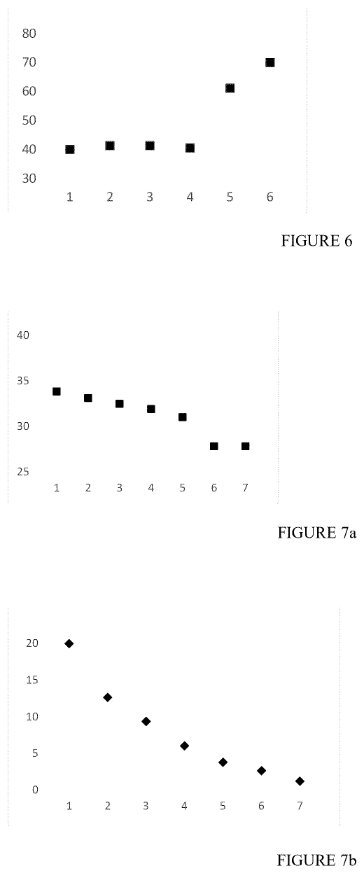

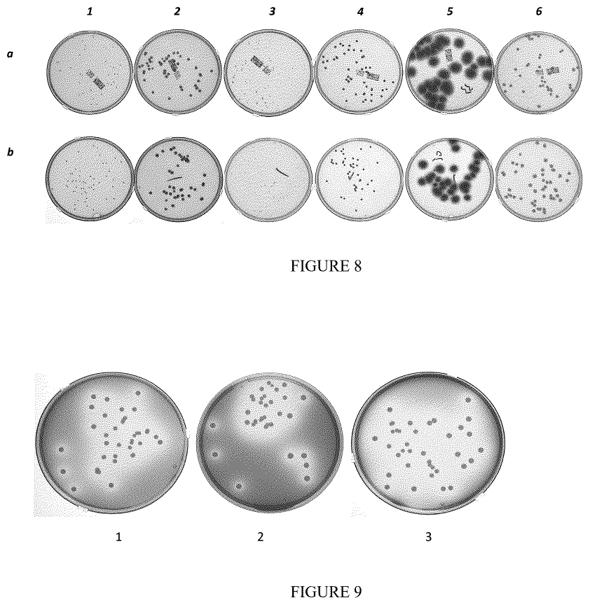

Conclusion: As may be seen from FIG. 1, all the media with 100% agar (media 1a, 2a, 3a, 4a, 5a, 6a) show very poor shrinkage resistance. All the agars peel off the dish wall and shrink during the test. The use of an agar:κ-carrageenan mix (50:50) considerably improves the shrinkage resistance (media 1b, 2b, 3b, 4b, 5b, 6b).

Moreover, it was observed that the number of intact culture media post-exposure increased. The results are shown in FIG. 1. For example, for the European-Roko agar: κ-carrageenan mix (medium 4b), 43% of the culture media showed no shrinkage after the Extreme Dehydration Test. Moreover, the surface area of the dried culture media increased from 46.5% to 91.9%, as shown in Table 4.

Thus, the improvement in shrinkage resistance was observed on the agar:κ-carrageenan mix with all types of agar and agarose tested in this study, as shown in FIG. 1, FIG. 2 and Table 4.

### Example 3: Shrinkage-Resistance Testing of Media Comprising Different Carrageenans (Kappa, Lambda, Iota) and Culture Media According to the Invention

Different types of carrageenan were tested for shrinkage resistance:
κ-carrageenan (Setexam, Reference Danish agar: 03904187, Batch C19152)
κ-carrageenan (Roko, Reference Rokogel 4600: MV210120, Batch 210200659)
κ-carrageenan (Sigma-Aldrich, ref 22048, Batch BCCF0613)
ι-carrageenan (Sigma-Aldrich ref C1138, Batch SLCG0678)
λ-carrageenan (Sigma-Aldrich, ref 22049, batch BCBP8978V)

Each carrageenan was tested as a mixture with European agar (Setexam, ref. 03904185, Batch M2019). A ratio of agar:carrageenan of 50:50 was used. The TSA formulation was used for this test. The culture media were prepared using a Masterclave®. 30 ml of medium were added to each 90 mm Petri dish.

The Extreme Dehydration Test method as described in Example 2 was used to assess the shrinkage resistance of each medium in Petri dishes. The results were presented as a percentage calculated relative to the initial surface area of the culture medium.

TABLE 5
Results of the TSA Extreme Dehydration Test method with an
agar:carrageenan mix comprising different carrageenans.

| | | % Surface area |
|---|---|---|
| | | of dried |
| | | medium/surface area |
| | | of initial medium |
| Agar:Carrageenan = | 1 - European Agar | 46.5 |
| 100:0 | | |
| Agar:Carrageenan = | 2- κ-carrageenan -Sigma- | 80.0 |
| 50:50 | Aldrich | |
| | 3- κ-carrageenan -Setexam | 91.4 |
| | 4- κ-carrageenan -Roko | 94.8 |
| | 5- λ-carrageenan -Sigma- | 54.0 |
| | Aldrich | |
| | 6 ι-carrageenan -Sigma- | 54.2 |
| | Aldrich | |

As may be seen from Table 5 and FIG. 3, the use of an agar:κ-carrageenan mix improves the shrinkage resistance (media 2, 3, 4). This improvement was observed for all three κ-carrageenans tested from different suppliers. For example, for the agar:κ-carrageenan mix (Setexam), the surface area of the post-exposure culture medium is about 91% of the initial size, instead of 46% for the medium with agar. For the agar:κ-carrageenan (Sigma-Aldrich) mix and the agar:κ-carrageenan (Roko) mix, post-exposure agar-based surfaces areas are also improved.

However, mixing with A-carrageenan (medium 5) or t-carrageenan (medium 6) does not improve the shrinkage resistance of the medium. Over time, all the media shrink to become a small dried film with only 50% residual surface area relative to their initial surface area. In conclusion, improved shrinkage resistance was observed on mixes with all the κ-carrageenans tested in this study. In contrast, 2-carrageenan and t-carrageenan did not improve the shrinkage-related performance.

### Example 4: Shrinkage-Resistance Testing of Culture Media According to the Invention with Different Ratios of Agar: κ-Carrageenan

A series of ratios of agar:κ-carrageenan was tested to evaluate the shrinkage resistance (100:0, 70:30, 60:40, 50:50, 30:70, 0:100) with a total concentration of the gelling agent equal to 15 g/l. European agar (Setexam, ref 03904185, Batch M2019) and κ-carrageenan (Setexam, ref 03904187, Batch C19152) were used. The TSA formulation was used for this test. The culture media were prepared using a Masterclave®. 30 ml of media were added to each 90 mm Petri dish.

The Extreme Dehydration Test method as described in Example 2 was used to assess the shrinkage resistance of these media.

TABLE 6
Results of the TSA Extreme Dehydration Test
method with an agar:κ-carrageenan mix (ratio
100:0, 70:30, 60:40, 50:50, 30:70 and 0:100)

| | | % Dried surface area / initial |
|---|---|---|
| | Agar:κ-carrageenan | surface area |
| Medium 1 | 100:0 | 46.5 |
| Medium 2 | 70:30 | 73.9 |
| Medium 3 | 60:40 | 89.4 |
| Medium 4 | 50:50 | 91.8 |
| Medium 5 | 30:70 | 97.4 |
| Medium 6 | 0:100 | 90.5 |

The medium with 100% agar shows poor shrinkage resistance. All the media detach from the dish wall and shrink during the test. The surface area of the dried medium is only 46.5% relative to its initial surface area.

The use of an agar:κ-carrageenan mix considerably improves the shrinkage resistance relative to an agar-based medium with 100% agar.

With a ratio of agar:κ-carrageenan of 70:30, all the media shrink during the test. However, the average surface area of the dried medium is 73.9% relative to the initial surface area, instead of 46.5% for TSA with 100% agar as shown in Table 6.

With a ratio of agar:κ-carrageenan of 50:50 (medium 4), 43% of the media showed no shrinkage after the Extreme Dehydration Test, as shown in FIG. 4. The average surface area of the dried media is 91.8% relative to the initial surface area, as shown in Table 6. With a ratio of agar:κ-carrageenan of 30:70 (medium 5), 70% of the media show no shrinkage after the Extreme Dehydration Test, as shown in FIG. 4. The average surface area of the dried media is increased to 97.4% relative to the initial surface area, as shown in Table 6.

Conclusion: using the agar:κ-carrageenan mix significantly improves the shrinkage resistance of the medium. This improvement was observed on different ratios of agar: κ-carrageenan: 70:30; 60:40; 50:50; 30:70.

### Example 5: Gelling Test for Culture Media According to the Invention with Different Ratios of Agar:κ-Carrageenan

A series of ratios of agar:κ-carrageenan was tested (100:0, 80:20, 70:30, 60:40, 50:50, 30:70, 0:100) with a total concentration of the gelling agent equal to 15 g/l. European agar (Setexam) and κ-carrageenan (Setexam or Roko) were used with TSA, Chapman, and Sabouraud Dextrose Agar formulations. The solutions were autoclaved in a liquid cycle, with a plateau at 120° C. for 16 min, then placed in a water bath preheated to 80° C. The gelling temperature of each medium was measured using a Discovery HR-2 rheometer (TA Instruments).

The results obtained with the modified TSA medium are represented in FIGS. 5 (a) and 5 (b).

For the TSA medium with an agar:κ-carrageenan mix, formulations with ratios of 70:30, 50:50 and 30:70 have gelling temperatures of between 30° C. and 45° C. If the proportion of κ-carrageenan is increased by more than 70% (medium 5), the gelling temperature rises above 45° C., as shown in FIG. 5 (a). The viscosity of the medium also becomes very high, as shown in FIG. 5 (b)

Increasing the proportion of κ-carrageenan beyond 70% of the mix may make industrialization difficult. Modifications to the production process are required. Furthermore, the manufacture of media with heat-sensitive additives (antibiotics) or blood media (sheep or horse blood agar) also becomes impossible.

Conclusion: For the modified TSA medium or culture media with salt concentrations of between 5-20 g/l, it is preferable to have a κ-carrageenan proportion of 70% or less in the agar:κ-carrageenan mix.

The results obtained with the modified Chapman medium are represented in FIG. 6.

The gelling temperature for the Chapman medium with 100% agar is about 40° C. (medium 1). For the agar:κ-carrageenan mix, media with a ratio of 80:20 to 60:40 (media 2, 3, 4) have a gelling temperature similar to that of the Chapman medium.

Media with a 50:50 ratio or with a κ-carrageenan proportion greater than 50% (media 5 and 6) have a very high gelling temperature (>60° C.). This may make industrialization difficult. The manufacture of media with heat-sensitive additives (antibiotics) or blood media (sheep or horse blood agar) also becomes impossible.

Conclusion: For the Chapman medium or other culture media with high ion loads including a salt concentration of greater than 20 g/l, the use of an 80:20 to 60:40 ratio is preferred. The presence of ions accelerates the gelation of κ-carrageenan and increases the gelling temperature.

The results obtained on Sabouraud Dextrose Agar (SDA) medium are represented in FIG. 7.

The gelling temperature (FIG. 7a) for an SDA medium with 100% agar is about 34° C. (medium 1). For the agar:κ-carrageenan mix, media with a ratio of 80:20 to 50:50 (media 2 to media 5) have a gelling temperature of between 30° C. and 45° C. Media with a 30:70 ratio or with a κ-carrageenan proportion greater than 70% (media 6 and 7) have a very low gelling temperature (<30° C.), as shown in FIG. 7(a).

The elastic modulus of the medium (FIG. 7b) is also measured with the rheometer. For the agar:κ-carrageenan mix, media with a ratio of 80:20 to 60:40 (media 2, 3, 4) have an acceptable elastic modulus (>5 kPa). Media with a ratio of 50:50 or with a κ-carrageenan proportion of greater than 50 (media 5, 6, 7) are very soft. The media are not sufficiently solidified to be handled or inoculated.

Conclusion: For the SDA medium or other culture media with low ion loads and a salt concentration below 5 g/l, the use of a ratio of 80:20 to 60:40 is preferred.

### Example 6: Detection of Microorganisms Using Culture Media According to the Invention

In order to evaluate the microbiological performance of the media, tests of microorganism growth are performed using the strains listed in the table below. The strains used are obtained from BioBall® MultiShot 550.

Two dishes of each medium were inoculated with each microorganism and incubated as specified in the table below. After incubation, the dishes were counted using a Scan® 4000 automatic colony counter (ref 438000, supplier Interscience).

The recovery rate (RR) was calculated. It corresponds to the ratio of the number of colonies on test dishes to the number of colonies on control dishes with unmodified culture medium.

The desired recovery rate is 50 to 200%, as recommended by the Pharmacopeias.

The TSA formulation and the Chapman formulation were used for this test.

TABLE 7
Microorganism tested for TSA medium:

| | | | | Incu- | Incubation |
|---|---|---|---|---|---|
| Micro- | | Inoculum | | bation | temper- |
| organism | ATCC | Source | Inoculum | time | ature |
| Staphylococcus | ATCC | BioBall | BioBall: | 24 h ± 4 h | 30-35° C. |
| aureus | 6538 | Multishot | 100 μl | | |
| Pseudomonas | ATCC | 550 CFU | | | |
| aeruginosa | 9027 | | | | |
| Escherichia | ATCC | | | | |
| coli | 8739 | | | | |
| Bacillus | ATCC | | | | |
| subtilis | 6633 | | | | |
| Candida | ATCC | | | 48 h ± 6 h | |
| albicans | 10231 | | | | |
| Aspergillus | ATCC | | | | |
| brasiliensis | 16404 | | | | |

TABLE 8
Microorganism tested for the Chapman medium:

| | | | | Incu- | Incubation |
|---|---|---|---|---|---|
| Micro- | | Inoculum | | bation | temper- |
| organism | ATCC | Source | Inoculum | time | ature |
| Staphylococcus | ATCC | BioBall | BioBall: | 24 h-72 h | 30-35° C. |
| aureus | 6538 | Multishot | 100 μl | | |
| | | 550 CFU | | | |

The results are shown in the table below.

TABLE 9
Recovery rate of TSA with an agar:κ-carrageenan
mix (ratio 70:30, 50:50, and 30:70) relative
to control dishes with unmodified TSA medium.

| Microorganism | 70:30 | 50:50 | 30:70 |
|---|---|---|---|
| Staphylococcus aureus | 87% | 112% | 79% |
| Bacillus subtilis | 91% | 93% | 82% |
| Escherichia coli | 86% | 104% | 69% |
| Pseudomonas aeruginosa | 90% | 86% | 90% |
| Candida albicans | 75% | 83% | 88% |
| Aspergillus brasiliensis | 121% | 95% | 121% |

TABLE 10
Recovery rate of Chapman medium with an agar:κ-carrageenan
mix (ratio 80:20, 50:50, 60:40) relative to
unmodified Chapman medium.

| Microorganism | 80:20 | 50:50 | 60:40 |
|---|---|---|---|
| Staphylococcus aureus | 106% | 107% | 89% |

Conclusion: Media with an agar:κ-carrageenan mix show good detection and growth of microorganisms. The recovery rates are similar to those of unmodified media, and well in accordance with Pharmacopeia requirements, i.e. between 50%-200% for all microorganisms tested. The colony sizes and morphologies of dishes 1b to 6b (FIG. 8) inoculated respectively with the microorganisms mentioned in Table 9 on TSA medium with a ratio of agar:κ-carrageenan of 70:30, are also very similar to those of the control dishes (dishes 1a to 6a) comprising TSA medium with 100% agar. Likewise for Staphylococcus aureus colonies (FIG. 9) inoculated on Chapman medium with an agar:κ-carrageenan mix of 80:20 (dish 2) and 50:50 (dish 3) versus Chapman medium with 100% agar (dish 1).

### BIBLIOGRAPHICAL REFERENCES

Lines, “value of the K+ salt of carrageenan as an agar substitute in Routine bacteriological media”, Applied and Environmental Microbiology, December 1977, pages 637-639

1. A culture medium for detecting microorganisms, comprising
agar or agarose
κ-carrageenan
the weight ratio [agar, κ-carrageenan]:[other gelling agent] being greater than 70:30.

2. The culture medium as claimed in claim 1, also comprising peptones.

3. The culture medium as claimed in claim 1, wherein the concentration of the agar and κ-carrageenan mixture is between 10 g/l and 30 g/l.

4. The culture medium as claimed in claim 1, wherein the weight ratio of agar:κ-carrageenan is between 80:20 and 30:70.

5. The culture medium as claimed in claim 1, wherein the concentration of salts present in the medium is less than 5 g/l and the weight ratio of agar:κ-carrageenan is between 80:20 and 60:40.

6. The culture medium as claimed in claim 1, wherein the concentration of salts present in the medium is between 5 g/l and 20 g/l and the weight ratio of agar:κ-carrageenan is between 80:20 and 30:70.

7. The culture medium as claimed in claim 1, wherein the concentration of salts present in the medium is greater than 20 g/l and the weight ratio of agar:κ-carrageenan is between 80:20 and 60:40.

8. The culture medium as claimed in claim 1, wherein the culture medium also comprises a heat-sensitive compound.

9. The culture medium as claimed in claim 8, wherein the heat-sensitive compound is an antibiotic.

10. The culture medium as claimed in claim 8, wherein the heat-sensitive compound is blood.

11. The culture medium as claimed in claim 1, wherein it is gelled in a Petri dish.

12. A process for obtaining a culture medium as claimed in claim 1, the process comprising:
supercooling the compounds in the culture medium to mix them,
cooling to a first temperature above the gelling temperature of the culture medium,
filling Petri dishes with this culture medium in solution, and
allowing to cool to reach the gelling temperature to form a gelled culture medium, the gelling temperature being between 30° C. and 45° C.

13. A culture medium comprising an agar:κ-carrageenan mixture having a weight ratio of between 80:20 and 30:70 and a gelling temperature of between 30° C. and 45° C., the weight ratio [agar, κ-carrageenan]:[other gelling agent] being greater than 70:30.

14. An in vitro microbiological culture method, in which microorganisms liable to be present in a sample are inoculated in or on a culture medium in a Petri dish as described in claim 1.

15. A method for detecting a target microorganism in a sample liable to contain same, the method comprising the following steps:
placing the sample in contact with a gelled culture medium as claimed in claim 1,
incubating, and
detecting the presence of the microorganism.
